(19) United States
(12) Patent Application Publication
(10) Pub. No.: US 2017/0128242 A1
(43) Pub. Date: May 11, 2017

(54) STENT

(71) Applicant: ACANDIS GMBH & CO. KG, Pfinztal (DE)

(72) Inventor: Andreas DING, Karlsruhe (DE)

(73) Assignee: ACANDIS GMBH & CO. KG, PFINZTAL (DE)

(21) Appl. No.: 15/295,063

(22) Filed: Oct. 17, 2016

(30) Foreign Application Priority Data
Oct. 15, 2015 (DE) 20 2015 105 466

(51) Int. Cl. A61F 2/88

(52) U.S. Cl. CPC A61F 2/885; A61F 2310/00149; A61F 2230/0067; A61F 2230/0069; A61F 2210/0014; A61F 2310/00143; A61F 2310/00023; A61F 2250/0098

(57) ABSTRACT

The invention relates to a stent having a tubular woven structure (3) formed from wire strands (1, 2) interwoven with one another, wherein at least one first wire strand (1) is formed by at least two individual wires (1.1, 1.2) which run adjacently and contact one another, wherein the first wire strand (1) winds helically in a first direction around an axis of rotation of the woven structure (3) and crosses at least one second wire strand (2), which comprises at least one individual wire (2.1) and winds helically in a second direction around the axis of rotation of the woven structure (3), and wherein at least one individual wire (1.1) of the first wire strand (1) comprises an X-ray visible core material which is encased by a sheath material having a lower X-ray visibility than the core material.

The invention relates to a stent having a tubular woven structure formed from wire strands interwoven with one another, wherein at least one first wire strand is formed by at least two individual wires which run adjacently and contact one another, wherein the first wire strand winds helically in a first direction around an axis of rotation of the woven structure and crosses at least one second wire strand, which comprises at least one individual wire and winds helically in a second direction around the axis of rotation of the woven structure, and wherein at least one individual wire of the first wire strand comprises an X-ray visible core material which is encased by a sheath material having a lower X-ray visibility than the core material. Such a wire formed from core material and sheath material is referred to as DFT wire.

The advantage of the invention lies in the fact that the position of the stent during implantation thereof can be monitored as a result of the X-ray visible core material. During implantation, the stent can be detected over the entire length thereof, which facilitates the positioning, in particular in curved vessel portions. The amalgamation of at least two wires to form a first wire strand results at the same time in an improved stability of the woven structure.

The first wire strand can consist of just two individual wires in preferred embodiments. The second wire strand can consist of just one individual wire. It is generally the case that not all wire strands that are wound in the same direction around the axis of rotation of the woven structure must have the same number of individual wires. Rather, the first wire strands can extend in a different turn direction and can extend helically around the axis of rotation of the woven structure in a manner crossing one another. By way of example, four first wire strands can thus be provided, which are interwoven between the second wire strands, wherein the four first wire strands are wound in pairs in different directions around the axis of rotation of the woven structure.

In a preferred embodiment of the stent, provision is made so that at least two, in particular both wires of the first wire strand are formed as DFT wires, i.e. comprise an X-ray visible core material which is encased by a sheath material of lower X-ray visibility. This increases the X-ray visibility of the woven structure. The sheath material preferably comprises a shape-memory material, in particular a nickel-titanium alloy.

The woven structure can comprise a total of four, six, or ten, preferably, eight DFT wires. In particular, all individual wires of the first wire strand can comprise an X-ray visible core material which is encased by a sheath material having a lower X-ray visibility than the core material. It is particularly preferred when all individual wires, in particular all individual wires of all wire strands that form the woven structure, are formed as DFT wires.

The woven structure preferably has a conical or cylindrical outer contour. It is also possible for the woven structure to comprise a cylindrical portion and one or more conical or truncated cone-shaped portions. In particular, the woven structure can comprise truncated cone-shaped end portions which are connected to one another by a cylindrical portion.

Provision can also be made so that the wire strands form a deflection at a longitudinal end of the woven structure, wherein the wire strands are wound in a first direction around the longitudinal axis of the woven structure before the deflection, and wherein the wire strands are wound in a second, in particular opposite direction around the longitudinal axis of the woven structure after the deflection. The deflection fundamentally forms an end loop of the woven structure.

The cross-sectional area of the X-ray visible core material can account for approximately 20% to 40%, in particular 30% of the total cross-section of the DFT wire (core material plus sheath material) when the core material is platinum or platinum-iridium, and approximately 10% to 20% when the core material is tantalum.

Provision is preferably made so that, in the case of DFT wires which have a diameter of 35 μm (after polishing), approximately 14% to 16%, in particular 15.1%, of the cross-sectional area is occupied by the X-ray visible material, preferably a platinum-iridium alloy. In the case of DFT wires which have a diameter of 43 μm (after polishing), the cross-sectional area proportion of the X-ray visible material, in particular of the platinum-iridium alloy, is preferably approximately 27% to 29%, in particular 28.1%.

The stent, in particular the woven structure, is preferably suitable for feeding via a catheter having an inner diameter of approximately 0.42 mm (0.17 inches). The cross-sectional diameter of the stent in the compressed state is adapted accordingly. In the expanded state the stent can have a cross-sectional diameter of 3.5 mm. A stent of this type having an expanded cross-sectional diameter of 3.5 mm preferably consists of a total of 18 wire strands.

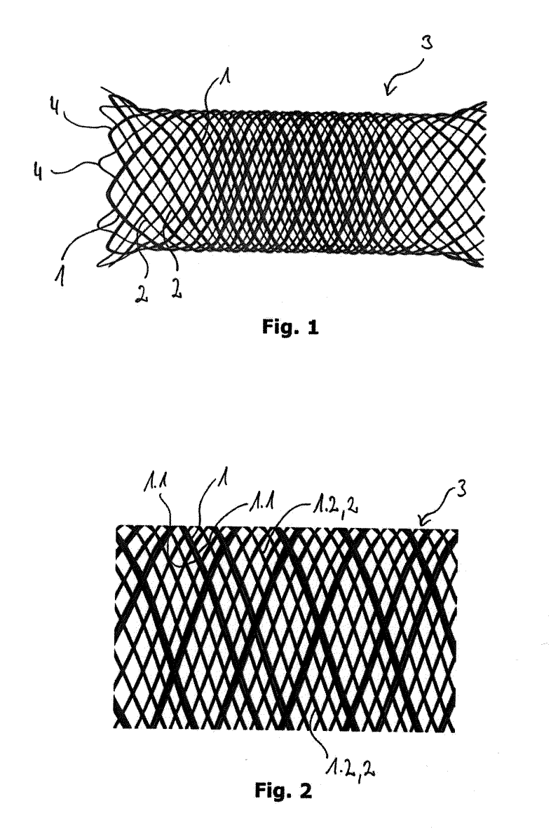

The wire strands can be deflected or can form a deflection at a longitudinal end of the woven structure and can be guided back in an opposite direction towards an opposite longitudinal end. Open wire ends are preferably arranged at an opposite longitudinal end of the woven structure (FIG. 1). Although wire strands are used to form the woven structure 18, the woven structure then appears to consist of twice the wire strand number, specifically 36 wire strands. It is also possible for the woven structure to appear to consist of 32 or 40 wire strands in order to form a stent having an expanded cross-sectional diameter of 3.5 mm, but in fact to be formed by the weaving and deflection of 16 or 20 wire strands.

In the case of a stent having an expanded cross-sectional diameter of 3.0 mm or 2.5 mm, the woven structure is preferably formed from 16 wire strands, which are deflected and woven back at a longitudinal end of the woven structure. The woven structure then appears to consist of 32 wire strands on account of the deflection of the wire strands at one end. Alternatively, the woven structure in the case of such a stent of which the expanded cross-sectional diameter is 3.0 mm or 2.5 mm can appear to consist of 24 or 36 strands, wherein in fact only 12 or 18 wire strands are required to form the woven structure and are deflected and woven back longitudinally axially.

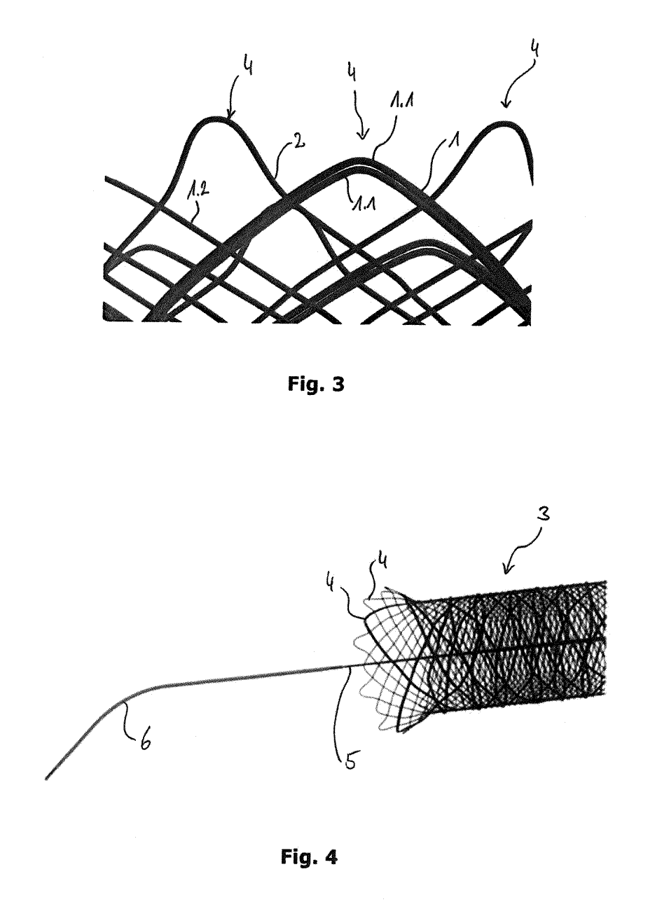

It is preferred when eight DFT wires on the whole are provided, which form a cross-sectional diameter between 40 μm and 45 μm, in particular 43 μm, and four first wire strands on the whole. Due to the deflection of the four wire strands at a longitudinal end of the woven structure, the woven structure appears to be formed from eight wire strands each having two DFT wires. The rest of the, in particular second, wire strands can each comprise a single individual wire having a cross-sectional diameter of 35 μm. This can be seen in FIGS. 2 and 3.

Provision can also be made so that the woven structure comprises two or six or ten first wire strands. The cross-sectional diameter of the individual wires not formed as DFT wires can generally be 35 μm or 30 μm or 40 μm. It is also conceivable for all individual wires to be embodied as DFT wire.

The used weave type can be 1-over-2. This means that one wire strand wound in a first direction around a longitudinal axis of the woven structure crosses over two wire strands and crosses under the next two wire strands. The 1-over-2 weave type can be seen in FIG. 2 and FIG. 3.

The weave angle, i.e. the angle at which the wire strands wound in opposite directions around the axis of rotation of the woven structure cross one another is preferably 140° in a middle portion of the woven structure. Each wire strand thus encloses an angle of 70° with the longitudinal axis of the woven structure. At the transition to a deflection or end loop at a longitudinal end of the woven structure, the weave angle is preferably approximately 130°. At this point, the corresponding wire strand thus encloses an angle of 65° with the longitudinal axis of the woven structure.

Crimped sleeves can be provided as additional X-ray markers at the ends. One, two, or three X-ray markers are advantageously arranged at each longitudinal end of the woven structure. The X-ray markers can be formed by crimped sleeves, which are each connected in a crimped manner to an individual wire or a wire strand formed from a plurality of individual wires, in particular two individual wires.

The stent preferentially has a length between 10 mm and 25 mm, preferably between 15 mm and 20 mm.

The stent is suitable for treatment, in particular for flow isolation, of aneurysms in arteries. The surface of the wires is preferably coated so as to be resistant to corrosion. One or both stent ends or weave ends can be shaped atraumatically by forming end loops. The stent preferably can be retracted into a catheter. One or both stent ends or weave ends can comprise a flared portion, i.e. a diameter enlargement (truncated cone-shaped widening).

In order to feed the stent into a blood vessel, the stent, or in particular the woven structure, can be connectable to a transport wire. The connection can be produced in particular in a form-fitting manner. The connection can be designed such that the stent, in the compressed state, is fixed in a form-fitting manner to the transport wire, wherein the connection automatically detaches when the stent expands. The transport wire preferably has a flexible tip. The flexible tip can also be bent or curved so as to act in an atraumatic manner. This is shown by way of example in FIG. 4.

The accompanying drawings show the following:

FIG. 1 shows a side view of a stent in accordance with a preferred exemplary embodiment of the present invention;

FIG. 2 shows a detailed view of a middle portion of the woven structure of the stent according to FIG. 1;

FIG. 3 shows a detailed view of an end portion of the woven structure of the stent according to FIG. 1; and

FIG. 4 shows a perspective partial view of the stent according to FIG. 1 with a transport wire for feeding the stent into a blood vessel.

The reference numerals in the drawings are associated with the following components/elements of the invention:
“1” denotes a first wire strand of the woven structure
“2” denotes a second wire strand of the woven structure,
“1.1” denotes a first individual wire formed as DFT wire
“1.2” denotes a second individual wire formed as DFT wire,
“3” denotes the woven structure,
“4” denotes a deflection or end loop,
“5” denotes a transport wire, and
“6” denotes a flexible, curved tip of the transport wire.

1. A stent having a tubular woven structure (3) formed from wire strands (1, 2) interwoven with one another, wherein at least one first wire strand (1) is formed by at least two individual wires (1.1, 1.2) which run adjacently and contact one another, wherein the first wire strand (1) winds helically in a first direction around an axis of rotation of the woven structure (3) and crosses at least one second wire strand (2), which comprises at least one individual wire (2.1) and winds helically in a second direction around the axis of rotation of the woven structure (3), and wherein at least one individual wire (1.1) of the first wire strand (1) comprises an X-ray visible core material which is encased by a sheath material having a lower X-ray visibility than the core material.

2. The stent according to claim 1,
characterised in that
at least two, in particular both individual wires (1.1, 1.2) of the first wire strand (1) comprise an X-ray visible core material which is encased by a sheath material having a lower X-ray visibility than the core material.

3. The stent according to claim 1 or 2,
characterised in that
all individual wires (1.1, 1.2) of the first wire strand (1) comprise an X-ray visible core material which is encased by a sheath material having a lower X-ray visibility than the core material.

4. The stent according to any one of the preceding claims,
characterised in that
the individual wires (1.1, 1.2) of all wire strands (1, 2) comprise an X-ray visible core material which is encased by a sheath material having a lower X-ray visibility than the core material.

5. The stent according to any one of the preceding claims,
characterised in that
the woven structure (3) has a conical or cylindrical outer contour.

6. The stent according to any one of the preceding claims,
characterised in that
the wire strands (1, 2) form a deflection at a longitudinal end of the woven structure, wherein the wire strands (1, 2) are wound in a first direction around the longitudinal axis of the woven structure (3) before the deflection (4), and wherein the wire strands (1, 2) are wound in a second direction around the longitudinal axis of the woven structure (3) after the deflection (4).

7. The stent according to any one of the preceding claims, characterised in that
the sheath material comprises a shape-memory material, in particular a nickel-titanium alloy.
